(19) United States
(12) Patent Application Publication
(10) Pub. No.: US 2015/0313659 A1
(43) Pub. Date: Nov. 5, 2015

(54) FASTENING FORCE AUXILIARY DEVICE FOR SCREW AND SCREW WITH FASTENING FORCE AUXILIARY DEVICE

(71) Applicants: KAGOSHIMA UNIVERSITY, Kagoshima-shi, Kagoshima (JP); MARUYAMA IRONWORKS CO., LTD., Kagoshima-shi, Kagoshima (JP)

(72) Inventors: Shouichi MIYAWAKI, Kagoshima-shi, Kagoshima (JP); Takakazu YAGI, Kagoshima-shi, Kagoshima (JP); Hiroshi TOMONARI, Kagoshima-shi, Kagoshima (JP); Yuichi KIMURA, Kagoshima-shi, Kagoshima (JP); Yasuo OSAKO, Kagoshima-shi, Kagoshima (JP); Satoshi MARUYAMA, Kagoshima-shi, Kagoshima (JP)

(21) Appl. No.: 14/650,280

(22) PCT Filed: Dec. 9, 2013
(86) PCT No.: PCT/JP2013/082954

§ 371 Date: Jun. 5, 2015

(30) Foreign Application Priority Data
Dec. 7, 2012 (JP) 2012-268204

(51) Int. Cl. A61B 17/86

(52) U.S. Cl. CPC A61B 17/8695; A61B 2017/8655

(57) ABSTRACT

A fastening force auxiliary device (1) includes a seat part (10) in which a hole (11) allowing a screw part (3) of a miniscrew (2) to be inserted through is formed and which receives a head part (5) of the miniscrew (2). Further, the fastening force auxiliary device (1) has three spiked portions (13) extending from a periphery of the seat part (10) in a driving direction of the miniscrew (2). The spiked portions (13) extend so as to spread outward from the periphery of the seat part (10) and so as to gradually taper off. When the miniscrew (2) is implanted and fixed in a jaw bone or the like in an oral cavity, it is screwed into the jaw bone or the like while the screw part (3) is inserted through the hole (11) of the fastening force auxiliary device (1). Consequently, a flange (7) is received by the seat part (10) and tips of the spiked portions (13) come into pressure contact with a surface of the jaw bone or the like by being pressed by the flange portion (7). Consequently, it is possible to greatly reduce the failure of the miniscrew (2).

### TECHNICAL FIELD

The present invention relates to a fastening force auxiliary device for screw and a screw with a fastening force auxiliary device which are suitable for use in, for example, orthodontic treatment.

### BACKGROUND ART

In orthodontic treatment, in order to move a target tooth by an orthodontic force, an anchor which resists the orthodontic force is required. As the anchor, another tooth (molar or the like), the head, the cervix, or the like has been generally used.

However, the use of the other tooth as the anchor has a drawback that a tooth not intended to be moved sometimes moves. Further, the use of the head or the cervix as the anchor has a drawback that a patient's cooperation is indispensable since an effect is not obtained unless an appliance such as a headgear is worn.

After Branemark developed a titanium dental root (dental implant) with high bio-compatibility which exhibits firm osseo-integration, orthodontic treatment using this as an absolute anchor was devised in the 1960s and has come into use.

However, the titanium dental implant is very expensive and can be implanted in a limited region, and in addition, its surgical invasion is large, and further, due to its firm integration with the bone, its removal after the implantation is difficult and thus it is not easily used for reinforced anchorage for temporary use, and for these reasons, orthodontic treatment using, as the anchor, a miniscrew which has been used for anchoring bone fragments in the treatment of bone fracture or the like has recently come into practice (refer to Patent Literature 1 and Patent Literature 2, for instance). This art is to implant and fix the miniscrew in, for example, a jaw bone, an alveolar bone, a buccal bone, or a palatal bone (hereinafter, referred to as “a jaw bone or the like) in an oral cavity and fix a plate and a wire by supporting them by this miniscrew.

### CITATION LIST

### Patent Literature

Patent Literature 1: Japanese Laid-open Patent Publication No. 11-164843

Patent Literature 2: Japanese Laid-open Patent Publication No. 2001-187071

### SUMMARY OF INVENTION

### Technical Problem

The use of the miniscrew needs only a small surgical invasion and can also reduce an uncomfortable symptom of a patient. A recent mainstream approach is a self-drilling type which more facilitates the implantation (which makes it possible to directly implant the screw without making a hole in a bone in advance).

However, the miniscrews always involve a risk of their contact with dental roots because the miniscrews have to be accurately implanted in a narrow space between a dental root and a dental root in an alveolar bone at the time of the implantation. The miniscrews further have drawbacks that they can be implanted in limited regions and applied to limited disease cases, damage dental roots, require advanced diagnostic and implantation technologies, and 15% of them failure. Besides the form of the miniscrew, an implantation operation requiring a skill, and so on, possible causes for making the miniscrew failure are the contact of the miniscrew with a dental root in a jaw bone or the like. In particular, when the miniscrew has a long length, its portion penetrating through a cortical bone (about 1 to 3 mm superficial portion of the bone) of the jaw bone or the like to enter a cancellous bone becomes long, and accordingly the risk of the contact with a dental root, which is a main cause of the failure, increases.

Further, a possible measure to enhance the percentage of success of the implantation may be to increase a physical contact area between the miniscrew and the cortical bone of the jaw bone or the like to thereby increase a fastening force, but increasing the diameter of the miniscrew not only increases the risk of its contact with the dental root, which is the main cause of the failure, but also may cause a case where a crack runs through the cortical bone due to an increase of a torque at the time of the implantation in the self-drilling.

Therefore, when the conventional miniscrew is used, it is necessary to decide safe position and insertion angle at the time of the implantation in order avoid its contact with a dental root, which is disadvantageous in that expensive image inspection such as CT and accompanying exposure to radiation, and further advanced diagnostic technology and advanced treatment technology are required.

In addition, it has not been possible to use the conventional miniscrew for a patient (child) in a mixed dentition period because a tooth germ of a permanent tooth exists in his/her alveolar bone and thus the tooth germ is damaged when the miniscrew is implanted by an ordinary operative method. Therefore, the guideline of anchor screws for orthodontic treatment prepared by Japanese Orthodontic Society in September, 2012 limits the applicable age to, in principle, the adult or the youth in the last growth stage after his/her permanent dentition is completed.

For example, a miniscrew or the like whose pitch is set differently on thread portions that come into contact with a cortical bone and a cancellous bone of a jaw bone or the like in order to acquire stability of the miniscrew has been contrived. Attempts to improve the percentage of success have been thus made, but there is data that about 15% failure at a region other than a palate, and a solution has not been obtained yet.

As described above, to obtain stability of the miniscrew after its implantation, the miniscrew is required to have length and diameter that are long to a certain degree, but on the contrary, this may lead to the contact with a dental root and a decrease of a fastening force, and they are in a trade-off relation. For these reasons, it is the current situation that, even though an improvement of the conventional miniscrew is progressing, there still exists the limitation of an implantable area and the risk of the failure and breakage cannot be eliminated yet.

The present invention was made in consideration of the aforesaid points, and has an object to greatly reduce the failure of a screw by dramatically enhancing safety and a fastening force of the screw and to widen the range of applications regarding an implantable area.

### Solution to Problem

A fastening force auxiliary device for screw of the present invention is a fastening force auxiliary device for screw for retaining a fastening force of a screw implanted in a target region, the auxiliary device including: a seat part in which a hole allowing a screw part of the screw to be inserted through is formed and which receives a head part of the screw; and a plurality of spiked portions extending from a periphery of the seat part in a driving direction of the screw, wherein, when the screw is implanted into the target region while the screw part is inserted through the hole, the head part is received by the seat part and tips of the plural spiked portions come into pressure contact with the target region by being pressed by the head part.

Further, another characteristic of the fastening force auxiliary device for screw of the present invention is that the tips of the spiked portions are pointed and penetrate in the target region instantaneously or over time.

Further, another characteristic of the fastening force auxiliary device for screw of the present invention is that the adjacent spiked portions are connected by a reinforcing part.

Further, another characteristic of the fastening force auxiliary device for screw of the present invention is that the screw is used as a very firm anchor when an orthodontic force for moving a tooth is applied.

A screw with a fastening force auxiliary device of the present invention is a screw implanted in a target region, the screw including a fastening force auxiliary device including: a seat part in which a hole allowing a screw part of the screw to be inserted through is formed and which receives a head part of the screw; and a plurality of spiked portions extending from a periphery of the seat part in a driving direction of the screw, wherein, when the screw is implanted into the target region while the screw part is inserted through the hole, the head part is received by the seat part and tips of the plural spiked portions come into pressure contact with the target region by being pressed by the head part.

Further, another characteristic of the screw with the fastening force auxiliary device of the present invention is that a biasing member which biases the fastening force auxiliary device toward the target region is provided between the screw and the fastening force auxiliary device. In this case, a stopper which restricts an amount by which the fastening force auxiliary device is moved by the biasing member may be provided.

### Advantageous Effects of Invention

According to the present invention, by the fastening force auxiliary device, it is possible to dramatically enhance the fastening force of the screw, widen the range of applications regarding an implantable area, and further greatly reduce the failure of the screw implantation.

### BRIEF DESCRIPTION OF DRAWINGS

FIG. 1A is a plane view of a fastening force auxiliary device for screw according to a first embodiment.

FIG. 1B is a side view of the fastening force auxiliary device for screw according to the first embodiment.

FIG. 1C is a bottom view of the fastening force auxiliary device for screw according to the first embodiment.

FIG. 2 is a perspective view of the fastening force auxiliary device for screw according to the first embodiment.

FIG. 3 is a perspective view of a miniscrew according to the first embodiment.

FIG. 4 is a view illustrating a use state of the miniscrew and the fastening force auxiliary device according to the first embodiment.

FIG. 5 is a perspective view illustrating a screw with a fastening force auxiliary device according to a second embodiment.

FIG. 6A is a plane view of a fastening force auxiliary device for screw according to a third embodiment.

FIG. 6B is a side view of the fastening force auxiliary device for screw according to the third embodiment.

FIG. 6C is a bottom view of the fastening force auxiliary device for screw according to the third embodiment.

FIG. 7 is a perspective view of the fastening force auxiliary device for screw according to the third embodiment.

FIG. 8 is an explanatory view of a screw with a fastening force auxiliary device according to a fourth embodiment.

FIG. 9 is an explanatory view of a modification example of the screw with the fastening force auxiliary device according to the fourth embodiment.

FIG. 10A is a view illustrating an example of the miniscrew.

FIG. 10B is a view illustrating an example of the miniscrew.

FIG. 10C is a view illustrating an example of the miniscrew.

FIG. 10D is a view illustrating an example of the miniscrew.

### DESCRIPTION OF EMBODIMENTS

Hereinafter, preferred embodiments of the present invention will be described with reference to the attached drawings.

### First Embodiment

The inventor of the present application has determined that it is not enough only to aim at improvement of only a miniscrew and has invented “a fastening force auxiliary device for screw” being its assisting device.

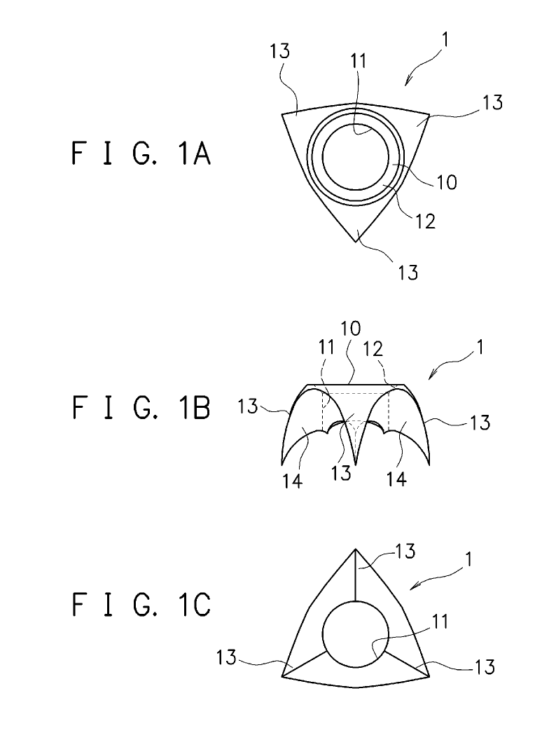

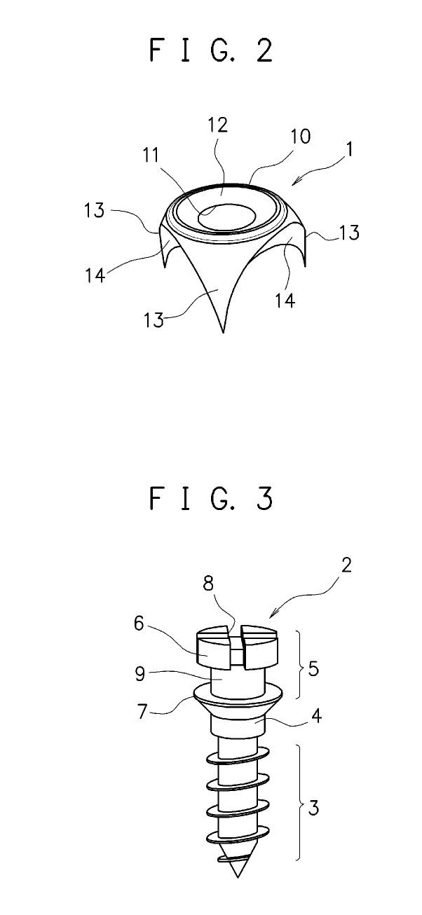

FIG. 1A to FIG. 1C and FIG. 2 illustrate a fastening force auxiliary device 1 for screw according to a first embodiment to which the present invention is applied. Further, FIG. 3 illustrates a miniscrew 2.

In orthodontic treatment, the miniscrew 2 illustrated in FIG. 3 is implanted in a jaw bone or the like in an oral cavity and is used as an anchor when an orthodontic force for moving a tooth is applied.

The miniscrew 2 has a screw part 3, a columnar part 4, and a head part 5 in order from a lower side. The screw part 3 is implanted in the jaw bone or the like and the head part 5 is partly exposed to the outside in the oral cavity. The head part 5 has an engagement portion 6 on its upper end side and a flange portion 7 located above the screw part 3. The engagement portion 6 has a substantially hexagonal prismatic shape and in its upper surface, a cruciform groove 8 is formed. It is possible to drive the screw part 3 by fitting a tool such a driver around a side surface of the engagement portion 6, or inserting a tool in the cruciform groove 8, and turning the tool while engaging the tool with the engagement portion 6. In the head part 5, a constricted portion 9 is formed between the engagement portion 6 and the flange portion 7. Orthodontic appliance and material are fixed by being connected to and supported by the constricted portion 9 and the engagement portion 6.

The fastening force auxiliary device 1 illustrated in FIG. 1A to FIG. 1C and FIG. 2 is used for increasing a retaining force of the miniscrew 2 implanted in the jaw bone or the like.

The fastening force auxiliary device 1 has a seat part 10 which receives the head part 5 of the miniscrew 2. At a center of the seat part 10, a hole 11 allowing the screw part 3 of the miniscrew 2 to be inserted through is formed. Here, a lower surface of the flange portion 7 of the miniscrew 2 is an arc surface in a tapered shape, and accordingly, the seat part 10 also has a concave portion 12 in an arc surface shape.

Further, the fastening force auxiliary device 1 has three led parts 13 extending from a periphery of the seat part 10 in a driving direction of the miniscrew 2. The spiked portions 13 extend so as to bulge out in an arc shape and spread outward from the periphery of the seat part 10 and so as to gradually taper off. The adjacent spiked portions 13 are connected by an arc surface part 14 which is dented inward, and this arc surface part 14 functions as a reinforcing part of the spiked portions 13.

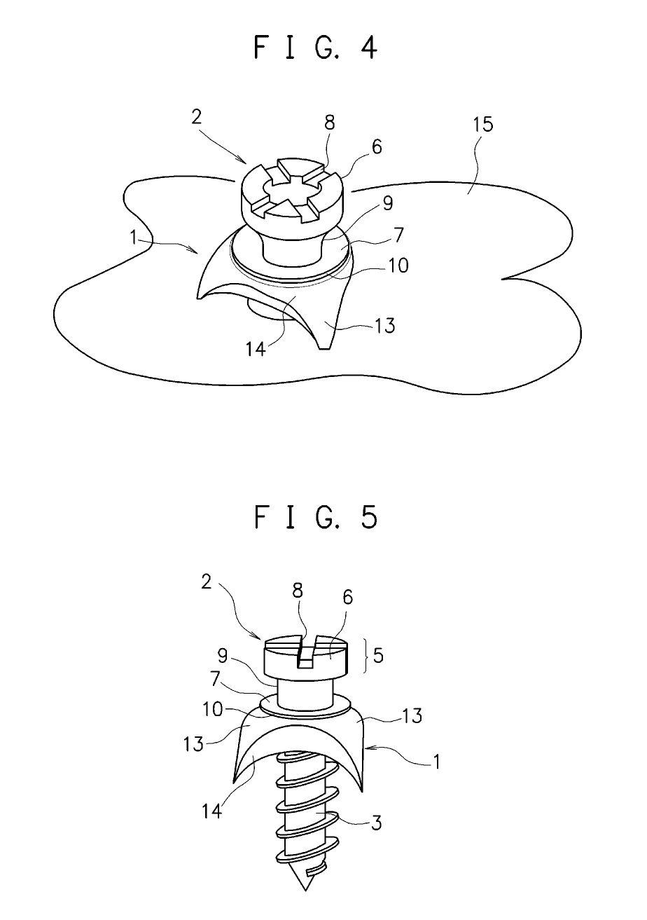

When the miniscrew 2 is implanted and fixed in the jaw bone or the like in the oral cavity, it is screwed into the jaw bone or the like while the screw part 3 is inserted through the hole 11 of the fastening force auxiliary device 1. Consequently, as illustrated in FIG. 4, the flange 7 is received by the seat part 10 and at the same time, tips of the three spiked portions 13 come into pressure contact with a surface 15 of the jaw bone or the like by being pressed by the flange portion 7. In this embodiment, the tips of the spiked portions 13 each have, for example, a triangular pyramid shape like a tip of a knife or a needle, or a shape like a sharp blade or needle pointed in a circular conic shape, and it is assumed that the tips of the spiked portions 13 penetrate by about 0.1 to 0.9 mm when a torque at the time of their implantation in a cortical bone of the jaw bone or the like becomes about 5 to 30 N. Note that the penetration amount is not limited and may be smaller than 0.1 mm or may be larger than 0.9 mm as required.

The use of the fastening force auxiliary device 1 according to this embodiment can dramatically improve the fastening force of the miniscrew 2, and increases a contact area with the cortical bone having high hardness to increase a mechanical fitting force, which can greatly reduce the failure of the miniscrew 2.

Specifically, since the spiked portions 13 of the fastening force auxiliary device 1 come into pressure contact with the surface (dense and high-strength cortical bone) of the jaw bone or the like to penetrate in the cortical bone, the retaining force of the miniscrew 2 in pressure contact with the fastening force auxiliary device 1 can be dramatically increased.

Further, since an orthodontic appliance is connected to the miniscrew 2, a tensile force in one radial direction acts. In this case as well, the spiked portions 13 of the fastening force auxiliary device 1 exhibits a resistive force against the tensile force. The spiked portions 13 can exhibit a large resistive force against the tensile force, especially by having the shape extending so as to spread outward, that is, so as to spread outward in the radial direction of the miniscrew 2 as in this embodiment. Therefore, it is possible to prevent the miniscrew 2 from being inclined or failure when a load of the orthodontic force during the treatment is applied.

As described above, since the retaining force of the miniscrew 2 can be dramatically improved, it is possible to use a short miniscrew that has not been conventionally usable, not to mention a conventionally available miniscrew (about 1.2 to 2.0 diameter, about 4.0 to 8.0 mm length). This not only makes it possible to avoid the contact with a dental root in the jaw bone or the like but also eliminates needs for expensive inspection such as CT, exposure to radiation, advanced diagnostic technology, and advanced treatment technology. Further, superiority over the conventional art lies in that applicable disease cases in which the miniscrew is usable for the youth in a deciduous dentition period and in a mixed dentition period increase.

Further, the fastening force auxiliary device 1 to which the present invention is applied is structured such that the tips of the three spiked portions 13 come into pressure contact with the surface of the jaw bone or the like to penetrate in the cortical bone, a reason for which is as follows.

Specifically, the surface of the jaw bone or the like is not always a flat surface and has a complicated shape having irregularities. Therefore, assuming that, for example, a washer in a disk shape is used, a gap is formed between the washer and the surface of the jaw bone or the like. On the other hand, in the fastening force auxiliary device 1 to which the present invention is applied, since the tips of the spiked portions 13 are sharp and thus the spiked portions 13 differently penetrate, it is possible to surely bring the tips of the three spiked portions 13 into contact with the surface of the jaw bone or the like even when the surface of the jaw bone or the like has a complicated shape having irregularities.

Further, the jaw bone or the like is covered by the gum in the oral cavity. Therefore, assuming that, for example, a disk-shaped washer is used, the washer presses the gum to intercept blood flow, which may cause necrosis or the like. Or, treatment with large surgical invasion such as exfoliating a wide area (corresponding to the area of the washer) of the gum is required. On the other hand, the fastening force auxiliary device 1 to which the present invention is applied does not have such problems because it is only necessary to bring the tips of the three spiked portions 13 into pressure contact with the surface of the jaw bone or the like.

### Second Embodiment

As a second embodiment, “a screw with a fastening force auxiliary device” in which the fastening force auxiliary device 1 and the miniscrew 2 described in the first embodiment are integrated will be described.

FIG. 5 illustrates the screw 2 with the fastening force auxiliary device 1 according to the embodiment to which the present invention is applied. Note that the same constituent elements as those of the first embodiment will be denoted by the same reference signs and a detailed description thereof will be omitted.

A failure preventing member is provided from a screw part 3 side so as to prevent the fastening force auxiliary device 1 from failure the miniscrew 2 and so as to allow the miniscrew 2 and the fastening force auxiliary device 1 to relatively rotate. Incidentally, owing to the presence of the flange portion 7, the fastening force auxiliary device 1 does not fail in a direction of the head part 5 of the miniscrew 2.

### Third Embodiment

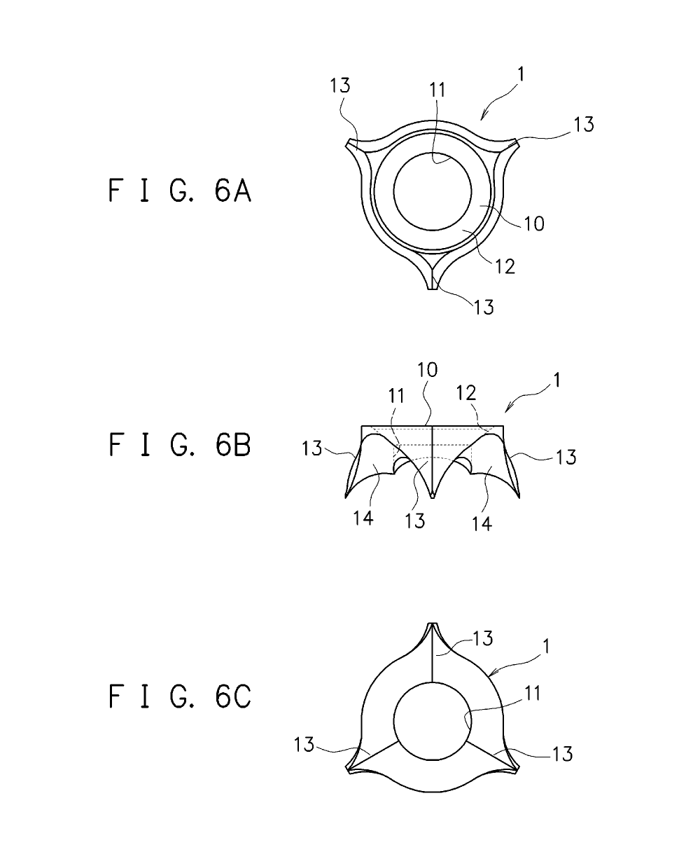

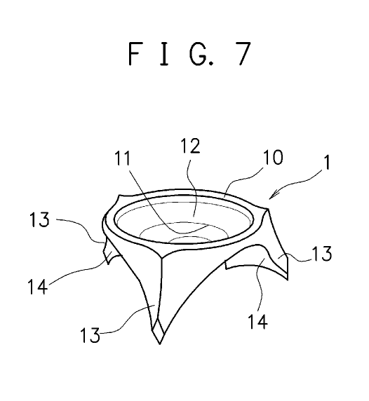

FIG. 6A to FIG. 6C and FIG. 7 illustrate a fastening force auxiliary device 1 for screw according to a third embodiment to which the present invention is applied. Note that its basic structure is the same as that of the first embodiment, with only its shape being different, and therefore, the same constituent elements will be denoted by the same reference signs and a detailed description thereof will be omitted.

In this embodiment as well, the fastening force auxiliary device 1 has a seat part 10 which receives a head part 5 of a miniscrew 2. At a center of the seat part 10, a hole 11 allowing a screw part 3 of the miniscrew 2 to be inserted through is formed. Here, a lower surface of a flange portion 7 of the miniscrew 2 is an arc surface in a tapered shape and accordingly the seat part 10 also has a concave portion 12 in an arc surface shape.

Further, the fastening force auxiliary device 1 has three spiked portions 13 extending from a periphery of the seat part 10 in a driving direction of the miniscrew 2. The spiked portions 13 extend so as to spread outward from the periphery of the seat part 10 and so as to gradually taper off. In the case of this embodiment, a ridgeline extending in an extension direction of the spiked portions 13 is formed at a center of each of the spiked portions 13. The adjacent spiked portions 13 are connected by an arc surface part 14 dented inward, and this arc surface part 14 functions as a reinforcing part of the spiked portions 13.

### Fourth Embodiment

As a fourth embodiment, “a screw with a fastening force auxiliary device” in which a fastening force auxiliary device 1 and a miniscrew 2 are integrated and which includes a biasing member will be described.

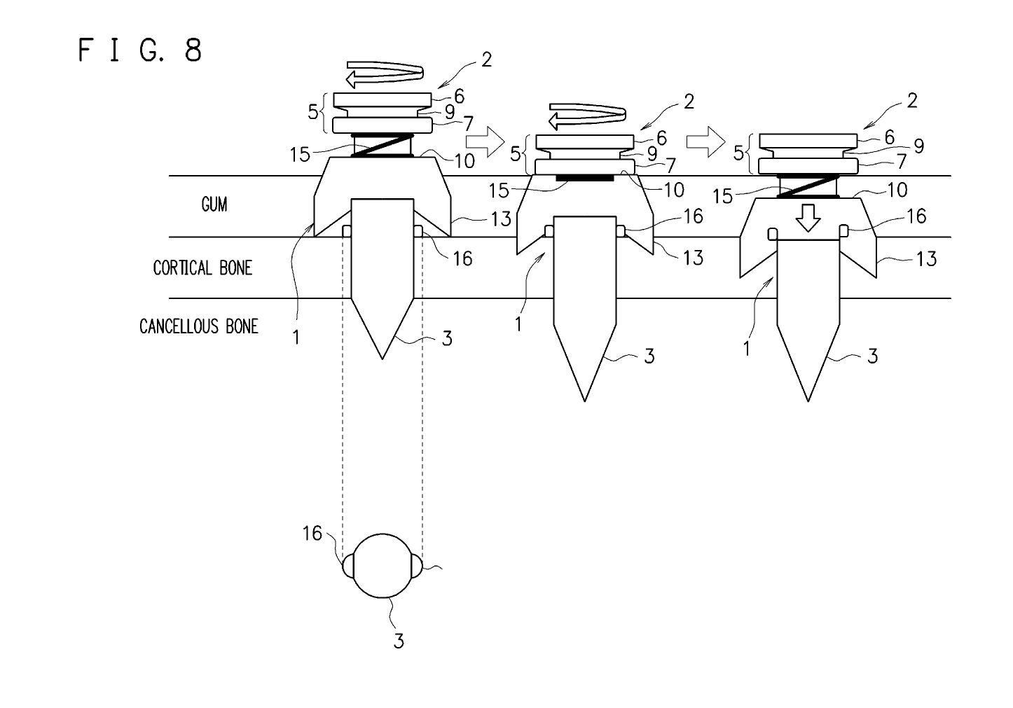

FIG. 8 illustrates the screw 2 with the fastening force auxiliary device 1 according to the embodiment to which the present invention is applied. Note that the same constituent elements as those of the first embodiment will be denoted by the same reference signs and a detailed description thereof will be omitted.

As illustrated in FIG. 8, the miniscrew 2 has a head part 5. Further, the fastening force auxiliary device 1 has: a seat part 10 in which a hole 11 (not illustrated in FIG. 8) allowing a screw part 3 of the miniscrew 2 to be inserted through is formed and which receives the head part 5 of the miniscrew 2; and a plurality of spiked portions 13 extending from a periphery of the seat part 10 in a driving direction of the miniscrew 2.

Incidentally, this embodiment illustrates one in which a flange portion 7 of the head part 5 of the miniscrew 2 has a flat shape, but the miniscrew 2 and the fastening force auxiliary device 1 may have the shapes described in the first to third embodiments.

In this embodiment, between the head part 5 of the miniscrew 2 and the seat part 10 of the fastening force auxiliary device 1, the biasing member 15 such as a leaf spring or a single-wound coil spring is movably sandwiched.

When the miniscrew 2 is implanted and fixed in a jaw bone or the like in an oral cavity, it is screwed into the jaw bone or the like while the screw part 3 is inserted through the hole 11 of the fastening force auxiliary device 1, as illustrated in the left state in FIG. 8. Consequently, as illustrated in the middle state in FIG. 8, the biasing member 15 is compressed and the flange 7 is received by the seat part 10, and tips of the spiked portions 13 come into pressure contact with a surface 15 of the jaw bone or the like by being pressed by the flange portion 7 to penetrate in a cortical bone. Then, after the time passes from the middle state in FIG. 8 (for example, four to eight weeks later), the biasing member 15 brings the tips of the spiked portions 13 into a state where they gradually penetrate more in the cortical bone of the jaw bone or the like, as illustrated in the right state in FIG. 8. Consequently, it is possible to exhibit a firm retaining force.

Here, the miniscrew 2 is equipped with a stopper 16. The stopper 16 restricts an amount by which the fastening force auxiliary device 1 is moved by the biasing member 15. By this stopper 16, it is possible to appropriately set a penetration amount of the tips of the spiked portions 13 in the cortical bone of the jaw bone or the like, which can prevent excessive penetration. Further, the stopper 16 also functions as a failure preventing member which is structured to prevent the fastening force auxiliary device 1 from failure the miniscrew 2 and to allow the miniscrew 2 and the fastening force auxiliary device 1 to relatively rotate. Incidentally, the shape and so on of the stopper 16 may be any.

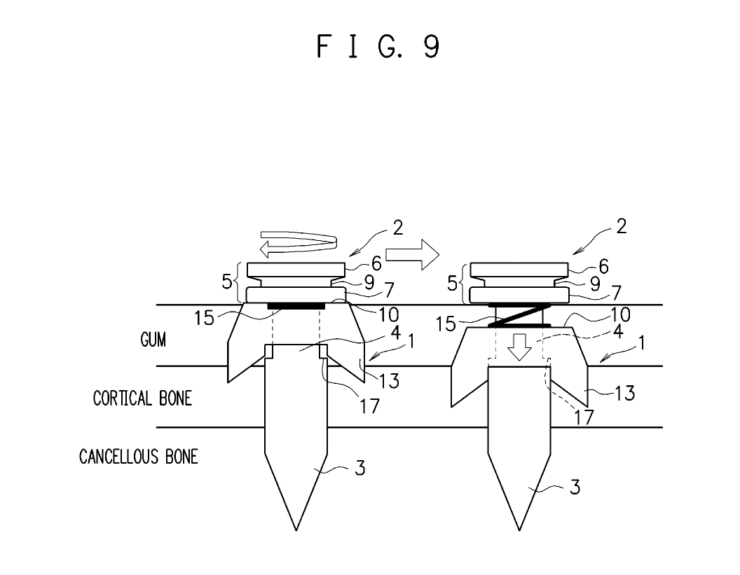

Further, the miniscrew 2 may be of a type not having the stopper 16 as a separate member. For example, as illustrated in FIG. 9, it may have a shape having a step 17, with a columnar part 4 between the head part 5 and the screw part 3 being smaller in diameter than an upper end of the screw part 3. The fastening force auxiliary device 1 is movable in a range of the columnar part 4 (range from a lower end of the head part 5 to the upper end of the screw part 3), but is not allowed to move more by the step 17 even when the fastening force auxiliary device 1 is biased by the biasing member 15. Incidentally, in FIG. 9, the step 17 is provided, but an inverse tapered shape with the columnar part 4 having a larger diameter as it goes toward the screw part 3 may be adopted. In this manner, the miniscrew 2 itself plays a role of the stopper which restricts the amount by which the fastening force auxiliary device 1 is moved by the biasing member 15. This form makes it possible to appropriately set the penetration amount of the tips of the spiked portions 13 in the cortical bone of the jaw bone or the like.

Hitherto, the preferred embodiments of the present invention are described, but the present invention is not limited to these embodiments, and various modifications and changes can be made therein within the scope of the spirit of the present invention.

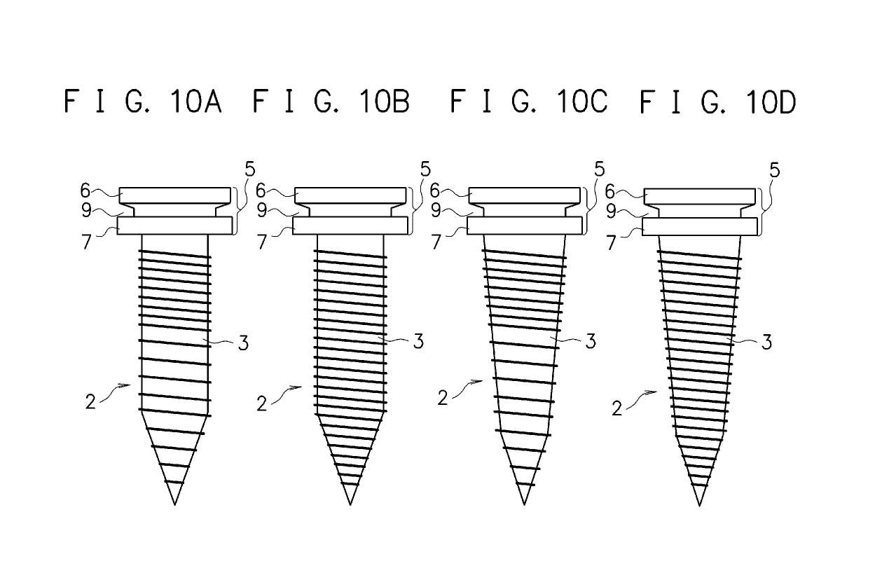

For example, the miniscrews 2 illustrated in the first to fourth embodiments are not limited, and various kinds of miniscrews are usable. FIG. 10A to FIG. 10D illustrate examples of the miniscrew 2. Those illustrated in FIG. 10A and FIG. 10B are types in each of which a screw part 3 is straight. That illustrated in FIG. 10A is a double-pitch miniscrew 2 in which a pitch on a portion screwed into the dense and high-strength cortical bone is fine and a pitch on a portion screwed into a cancellous bone is rough. That illustrated in FIG. 10B is a miniscrew 2 in which a pitch of a screw part 3 is uniform on the whole area. Further, those illustrated in FIG. 10C and FIG. 10D are tapered types in each of which a screw part 3 reduces in diameter toward its tip. That illustrated in FIG. 10C is a double-pitch miniscrew 2 in which a pitch on a portion screwed into the dense and high-strength cortical bone is fine and a pitch on a portion screwed into a cancellous bone is rough. That illustrated in FIG. 10D is a miniscrew 2 in which a pitch of a screw part 3 is uniform on the whole area.

Further, the example where the fastening force auxiliary device 1 has the three spiked portions 13 is described in the above embodiments, but their number is not limited. However, since three-point support or more increases stability more than two-point support, the number of the spiked portions 13 is preferably three or more. On the other hand, when the number of the spiked portions 13 becomes large, places pressing a gum increase or places exfoliating the gum increase, and therefore, their number is preferably three which is the minimum required number.

Further, the sizes and so on of the respective parts may be appropriately selected according to the thickness and so on of the gum and the cortical bone, and are not limited. Note that the height of the fastening force auxiliary device 1 is preferably low so as to reduce its portion protruding out of the gum in the oral cavity as much as possible.

### INDUSTRIAL APPLICABILITY

The present invention is usable even in a region where the implantation length of the screw cannot be sufficiently taken, and therefore is applicable as a substitute for a dental implant in a jaw bone, and is also applicable to the spinal cord requiring sufficient care for a nerve tract and other orthopedic regions, and further to general industrial products.

1. A fastening force auxiliary device for screw for increasing a retaining force of a screw while the screw is implanted in a cortical bone, the screw being used as an anchor when an orthodontic force for moving a tooth is applied, and implanted in a target region, the auxiliary device comprising:
a seat part in which a hole allowing a screw part of the screw to be inserted through is formed and which receives a head part of the screw; and
a plurality of spiked portions extending in a driving direction of the screw so as to spread outward from a periphery of the seat part and so as to gradually taper off; and in a driving direction of the screw, wherein,
a reinforcing part connection the adjacent spiked portions, wherein,
when the screw is implanted into the cortical bone target region while the screw part is inserted through the hole, the head part is received by the seat part and tips of the plural spiked portions come into pressure contact with the cortical bone target region by being pressed by the head part.

2. The fastening force auxiliary device for screw according to claim 1, wherein the tips of the spiked portions are pointed and penetrate in the cortical bone target region.

3-7. (canceled)

8. A screw with a fastening force auxiliary device which is a screw used as an anchor when an orthodontic force for moving a tooth is applied and implanted in a cortical bone, the screw comprising:
a fastening force auxiliary device including a seat part in which a hole allowing a screw part of the screw to be inserted through is formed and which receives a head part of the screw, a plurality of spiked portions extending in a driving direction of the screw so as to spread outward from a periphery of the seat part and so as to gradually taper off, and a reinforcing part connecting the adjacent spiked portions; and
a biasing member which is provided between the screw and the fastening force auxiliary device and biases the fastening force auxiliary device, wherein,
when the screw is implanted into the cortical bone while the screw part is inserted through the hole, the head part is received by the seat part and tips of the plural spiked portions come into pressure contact with the cortical bone by being pressed by the head part, from which state, the tips of the spiked portions are caused to penetrate in the cortical bone by the biasing member.

9. The screw with the fastening force auxiliary device according to claim 8, comprising a stopper which restricts an amount by which the fastening force auxiliary device is moved by the biasing member.
